(19) United States
(12) Patent Application Publication
(10) Pub. No.: US 2022/0243259 A1
(43) Pub. Date: Aug. 4, 2022

(54) DETERMINING THE DNA PRESENT IN A CELL BY IMAGING AND MATCHED FILTERING

(71) Applicant: THEMBA INC., SAN CARLOS, CA (US)

(72) Inventor: MATTHEW RABINOWITZ, SAN FRANCISCO, CA (US)

(73) Assignee: THEMBA INC., SAN CARLOS, CA (US)

(21) Appl. No.: 17/592,492

(22) Filed: Feb. 3, 2022

Related U.S. Application Data
(60) Provisional application No. 63/145,201, filed on Feb. 3, 2021.

(51) Int. Cl. C12Q 1/6827; C12Q 1/6883; G16B 30/10; G16B 20/20; G16B 20/10

(52) U.S. Cl. CPC C12Q 1/6827; C12Q 1/6883; C12Q 2600/156; G16B 20/20; G16B 20/10; G16B 30/10

(57) ABSTRACT

Disclosed herein are systems and methods for evaluating segments of DNA within a single strand or chromosome using matched filtering. DNA within a cell of interest, such as a gamete or embryonic cell, may be imaged at high resolution to provide an input signal. Matched filters may be created for reference signals from reference samples of cells having homologous chromosomes that share the same haplotypes as the DNA within the cell of interest. By applying a matched filter for a given haplotype to the input signal it can be determined whether the DNA within the cell of interest shares the same haplotype as the reference sample. The nucleotide sequence of one or more segments of DNA from the cell of interest may be reconstructed by identifying the haplotypes present in the DNA.

### CROSS-REFERENCE TO RELATED APPLICATIONS

This application claims the benefit of U.S. Provisional Application No. 63/145,201 filed on Feb. 3, 2021, which is herein incorporated by reference in its entirety.

### BACKGROUND

Genotyping is a valuable source of information for many applications in medicine, biotechnology, and assisted reproductive technology. Often, the genotype for a single cell is of particular interest, such as when the cell is a gamete or a cell from a developing embryo, where there is sparse genetic material available for standard sequencing methodologies. Additionally, single cell sequencing, even where there are additional cells of identical genome to evaluate can be expensive and/or inconvenient. Furthermore, many applications in which information on a genetic sequence would be useful require that the cell for which the genetic sequence is obtained remain healthy and viable for some application, such that there is a need for non-destructive genotyping technologies. Accordingly, there is a need for improved genotyping technologies that can effectively employ reference data from genetic sources sharing the same haplotypes as a cell of interest to genotype the cell of interest, optionally in a non-destructive manner.

### SUMMARY

The systems and methods disclosed herein involve creating matched filters based on nucleotide-derived signals obtained for one or more reference DNA samples comprising one or more known haplotype blocks that might also be present in the DNA of a separate cell of interest, and comparing those matched filters to a nucleotide-derived signal obtained from the DNA in the cell of interest in order to determine whether the one or more haplotype blocks are in fact present in the cell of interest (e.g., to determine which haplotype blocks from multiple candidate haplotype blocks, such as two candidate haplotypes from two homologous chromosomes, a DNA sequence in a cell of interest comprises). The haplotype blocks are genotyped using genetic samples large enough that the haplotype blocks can be reliably measured to create sufficiently accurate matched filters. Once it is determined what haplotype blocks are present in the cell of interest, by convolving the matched filter with the signal of the unknown DNA, the DNA sequence in the cell of interest can be known roughly as accurately as the candidate haplotype sequence is known. Although the resolution of the nucleotide-derived signal may not be sufficient to resolve the individual nucleotides of the DNA directly, it can be sufficient to enable determination of which haplotype blocks are present based on comparing the signal with a matched filter (e.g., by convolving the signal and matched filter).

In addition to nucleotide sequence, the systems and methods of the disclosure can also be used to determine de-novo copy number variants and aneuploidy from images of DNA in a cell. The approach works in the same way that a Code Division Multiple Access (CDMA) cell phone, or a GPS receiver, or radar, finds signals well below the noise floor, by comparing the incident noisy signal with a matched filter that has a replica of the code and achieves high processing gain when the code in the incident signal matches the reference code in the receiver.

According to one aspect of the disclosure, provided herein is a method of genotyping a segment of DNA. The method involves obtaining a signal derived from the DNA segment that is indicative of the nucleotide composition of the DNA segment and comparing the signal to one or more reference signals derived from different reference samples of DNA with matched filtering. Based on the matched filtering it is determined whether the nucleotide sequence of the DNA segment is substantially identical to a nucleotide sequence within one of the one or more reference samples.

Determining whether the nucleotide sequence of the DNA segment is substantially identical to a nucleotide sequence of one of the one or more reference samples may mean determining whether the DNA segment comprises the same haplotype block as the nucleotide sequence of one of the one or more reference samples. The DNA segment may be approximately the length of the haplotype block, less than the length of the haplotype block, or encompass multiple haplotype blocks. The haplotype block(s) may be at least about 100 kB in length. Comparing the signal to one or more reference signals may entail comparing the signal to two reference signals. Each of the two reference signals may be derived from two different but homologous chromosomes. Determining based on the matched filtering whether the nucleotide sequence of the DNA segment is substantially identical to a nucleotide sequence of one of the one or more reference samples may mean determining which of the two homologous chromosomes the DNA segment is derived from by determining which reference signal produces the highest output value from the matched filtering. The DNA segment may be a segment of a chromosome from a gamete.

Comparing the signal to one or more reference signals may entail comparing the signal to four reference signals. Each of the four reference signals may be derived from four different but homologous chromosomes. Determining based on the matched filtering whether the nucleotide sequence of the DNA segment is substantially identical to a nucleotide sequence of one of the one or more reference samples may mean determining which of the four homologous chromosomes the DNA segment is derived from by determining which reference signal produces the highest output value from the matched filtering. The DNA segment may be a segment of a chromosome from a diploid cell of an organism. Two of the four reference signals may be derived from a mother of the organism and two of the four reference signals may be derived from a father of the organism. The diploid cell may be an embryonic cell.

Comparing the signal to one or more reference signals with matched filtering may involve convolving the signal with conjugated reversed versions of each of the one or more reference signals.

The signal and one or more reference signals may be images of DNA. The images may be two-dimensional images or three-dimensional images. Obtaining a signal derived from the DNA segment that is indicative of the nucleotide composition of the DNA segment may entail performing the imaging of the DNA segment. The DNA within the images may be illuminated with a wavelength of light that preferentially distinguishes one type of nucleotide. The DNA within the images may be illuminated with multiple different wavelengths of light, such as, for example, 2, 3, or 4 wavelengths. Each wavelength may preferentially distinguish a different type of nucleotide. The DNA within the images may be stained with a fluorescent dye. The images of the DNA segment and images of the DNA within the reference samples may be taken with a single imaging apparatus.

The signal may be derived from the DNA segment in a live cell.

The aforementioned haplotype blocks of the one or more reference samples may be determined using long-read sequencing, synthetic long-read sequencing, or phasing based on parent genomes or population data.

The method may further entail assigning a nucleotide sequence to the DNA segment based on the nucleotide sequences of the one or more reference samples. The method may further entail determining a copy number of the DNA segment or of a portion of DNA with the DNA segment. Determining the copy number may involve identifying a copy number variant (CNV).

According to another aspect of the disclosure, provided herein is a method of genotyping a length of DNA having a plurality of segments. The genotyping is performed by genotyping each segment of the plurality of segments according to one of the aforementioned methods. The length of DNA may be a chromosome or single strand of DNA.

According to another aspect of the disclosure, provided herein is a method of screening a plurality of gamete cells. The method involves performing any one of the aforementioned methods for genotyping DNA on DNA within the plurality of gamete cells and selecting and isolating a gamete cell based on a genotype of one or more segments of DNA within the gamete cell.

The selected gamete cell may be disposed of or discarded. The selected gamete cell may be frozen and/or used in assisted reproduction, such as in an in vitro fertilization (IVF) procedure. The plurality of gamete cells may be sperms cells or ovum cells. Selecting and isolating a gamete cell based on a genotype of one or more segments of DNA within the gamete cell may entail generating one or more phenotype predictive models from the genotype.

According to another aspect of the disclosure, provided herein is a method of screening a plurality of embryos. The method involves performing any one of the aforementioned methods for genotyping DNA on DNA within at least one cell from each of the plurality of embryo and selecting and isolating an embryo based on a genotype of one or more segments of DNA within the embryo.

The selected embryo may be disposed of or discarded. The selected embryo may be frozen and/or used assisted reproduction, such as in an in vitro fertilization (IVF) procedure. Selecting and isolating an embryo based on a genotype of one or more segments of DNA within the embryo may entail generating one or more phenotype predictive models from the genotype.

According to another aspect of the disclosure, provide herein is a method of detecting chromosomal instability in tumor DNA. The method involves performing any one of the aforementioned methods of genotyping DNA on DNA within a tumor or cancer cell to determine a ploidy status for one or more chromosomal segments within the tumor or cancer cell.

Identification of an aneuploidy status for the one or more chromosomal segments is used to indicate chromosomal instability of at least some tumor cells.

The tumor or cancer cell may be from a subject diagnosed with cancer. The method may further entail treating the cancer or tumor cell or a subject from which the cell was obtained for cancer based on whether chromosomal instability has been indicated. The treatment may entail administering poly ADP ribose polymerase (PARP) inhibitors and/or platinum-based chemotherapeutics if chromosomal instability is indicated.

According to another aspect of the disclosure, provided herein is a method of generating signals indicative of the nucleotide composition of a segment of DNA within a cell of interest. The method involves imaging the segment of DNA within the cell of interest and imaging one or more homologous segments of DNA within one or more reference cells.

Images obtained from imaging the segment of DNA and one or more homologous segments of DNA may be two-dimensional or three-dimensional images. Imaging the segment of DNA and one or more homologous segments may involve illuminating the DNA with a wavelength of light that preferentially distinguishes one type of nucleotide. Illuminating the DNA may involve illuminating the DNA with multiple different wavelengths of light, such as, for example, 2, 3, or 4 wavelengths. Each wavelength may preferentially distinguish a different type of nucleotide. The segment of DNA and the one or more homologous segments of DNA may be stained with a fluorescent dye. The imaging of the segment of DNA and the one or more homologous segments of DNA may be performed with a single imaging apparatus.

According to another aspect of the disclosure, provided herein is a method involving creating a matched filter based on the known haplotype blocks of DNA that might be present in a cell. The method further entails comparing the matched filter to data from images made of the DNA in the cell and determining which haplotype blocks of DNA are present in the cell.

Determining the haplotype blocks may be done using long-read sequencing, synthetic long-read sequencing, or parent samples. One or more of the images made of the DNA may be achieved using high resolution photography. The method may further entail using the matching haplotype blocks to determine the sequences of nucleotides that are present in the cell. The method may further entail using the matching haplotype blocks to determine copy number and aneuploidy status in the cell. The images made may be of a plurality of sperm cells and the method may further entail using the determination of DNA in the plurality of sperm cells to generate phenotype predictive models from the DNA to select a sperm cell to use for fertilization of an egg.

### BRIEF DESCRIPTION OF THE FIGURES

FIGS. 1A-1B depict the UV absorbance (on a relative scale) of the nucleotides adenine, guanine, thymine, and cytosine (FIG. 1A) and a generic nucleic acid (FIG. 1B) at pH 7 across different wavelengths (nm).

FIG. 2 depicts alternative UV absorbance spectra for the nucleotides adenine, guanine, thymine, and cytosine.

FIG. 3 depicts the system architecture in one embodiment of the invention.

FIG. 4A depicts the convolution output and correlation peaks between the simulated imaged DNA and two possible reference homologs in red (correct homolog) and blue (incorrect homolog), where resolution is approximately 50 nm, the reference homolog length is 1 Mb, and the noise power is 10 times that of the signal power.

FIG. 4B depicts a zoom in on the correlation peaks in FIG. 4A.

FIG. 5A depicts the convolution output and correlation peaks for the matching (red) and non-matching (blue) homologs, where the resolution of the imaging is roughly 10 nm, the reference homologs are 200 kB, and the noise power roughly ten times the signal power.

FIG. 5B depicts a zoom in on the correlation peaks in FIG. 5A.

### DETAILED DESCRIPTION

The systems and methods described herein allow for the genotyping of a segment of DNA within a strand or chromosome of a cell of interest using a nucleotide-derived signal (e.g., an image) of the segment, one or more reference signals of DNA segments having one or more known genotype candidates, and one or more matched filters created from the reference signals, without the need for performing any sequencing on the DNA within the cell of interest. As used herein, genotyping may comprise the determination of a full or partial nucleotide sequence for the segment of DNA, the identification of one or more variants (e.g., alleles) in the segment of DNA, and/or the number of repeats or copy number of a segment of DNA or portion of a segment of DNA. The specificity of the type of genotyping possible depends on the genetic information that is available or known from the reference samples from which the one or more reference signals are derived. According to some aspects of the disclosure, genotyping a segment of DNA comprises determining a haplotype for the segment of DNA, based on one or more known haplotype candidates. As used herein, a segment of DNA may refer to any length or portion of a sequence of a single strand of DNA or chromosome that can be evaluated by the methods described herein, including an entire chromosome. According to various aspects of the disclosure, the cell of interest may be obtained from (e.g., isolated from) or may belong to a subject. A subject may refer to any organism having a genome, preferably a diploid genome. Preferably, the subject may be a mammal. According to various aspects of the disclosure, the subject is human. The methods described herein may be performed in vitro on a single cell. In some embodiments, the methods described herein may be performed on the cell in situ, such as, for example, within an embryo.

According to certain aspects of the disclosure, the cell of interest may be a sperm cell, oocyte cell, ovum cell, zygote, a single cell from a developing embryo, or a cancer or tumor cell. The cell of interest could be, for example, an ovum cell, in which case phased data from the female that made the ovum cell could be used to identify haplotypes and create matched filters, or a sperm cell, in which case phased data from the male that made the sperm cell could be used to identify haplotypes and create matched filters. The cell of interest could be, for example, a cell from a developing embryo, in which case phased data could be used from both parents to identify haplotypes and create matched filters. A cell of interest could be, for example, a cancer/tumor cell, in which case phased data from the host could be used to identify copy number errors or aneuploidies in the cell using the methods described herein.

“Phasing,” as used herein, may refer to the process of resolving the origin of haplotypes inherited from one of multiple homologous chromosomes. Phasing can be used, for example, to effectively resolve diploid DNA data, where it is not known to which homologous chromosome each nucleotide belongs, into haploid DNA data, where it is known what DNA sequencing, or haplotype block, is derived from each chromosome. Various methodologies for phasing such as synthetic long-read phasing using protocols like Universal Sequencing Technology, or long-read sequencing using systems like PacBio and Oxford Nanopore, or using parent(s), grandparent(s), or sibling(s) genetic data to resolve the phase of the child or parent, are well known in the art.

The cell of interest may have a segment of DNA having an unknown sequence. The unknown sequence may be known to be derived from one or more known DNA sequences, such that the unknown sequence is substantially identical to a known sequence or a combination of different portions of two or more sequences, comprising at least one known sequence (e.g., two or more known sequences). As used herein, a sequence of interest may be considered to be “derived” from (e.g., inherited from) a known sequence if the sequence of interest was generated by natural or artificial DNA-replicating processes performed on a source nucleic acid that has the same or substantially identical nucleotide sequence as the known sequence. Thus, for example, the sequence of interest and the known sequence may share a common genetic source or ancestor (e.g., they may share at least some of the same haplotypes)

The known sequences (reference sequence or reference codes) may be obtained from one or more reference samples as described herein. For example, the cell of interest may be a gamete (e.g., a sperm or an ovum) from a diploid organism, such as a human. The gamete may have an unknown DNA sequence which may be assumed to be identical to a sequence of one of two homologous chromosomes from the originating gametocyte, save any recombination events and/or germline mutations. The frequency of germline mutations may be assumed to be rare enough such that the unknown sequence is substantially identical to the sequence of one of the two homologous chromosomes, accounting for any recombination events. In other words, the unknown sequence may be presumed to be a combination of one or more haplotype blocks inherited from a parent cell (e.g., a gametocyte). A haplotype block generally comprises multiple variants such that the inheritance of one variant within a haplotype block infers the inheritance of the other variants within the same haplotype block. As used herein, a “variant” may refer to any difference between the sequence of two or more homologous chromosomes, including single nucleotide polymorphisms (SNPs), small insertions and deletions (Indels) and large copy number variants (CNVs). Alternatively, the cell of interest may be, for example, a single cell derived from a developing embryo. The unknown DNA sequence for an individual chromosome in the cell of interest may be presumed to be inherited from the embryo's mother or father. Accordingly, the unknown DNA sequence for the individual chromosome may be presumed to be a combination of one or more haplotype blocks inherited from the father or one or more haplotype blocks inherited from the mother. Alternatively, the cell of interest may be, for example, a cancer or tumor cell. The DNA sequence for an individual chromosome in the cell of interest may be presumed to be substantially identical to the DNA in non-cancer or non-tumor host cells, save for any somatic mutations. The frequency of most types of somatic mutations (e.g., point mutations, frameshift mutations) may be assumed to be rare enough such that the unknown sequence is substantially identical to the sequence of one of the two homologous chromosomes, accounting for any large scale structural mutations to the chromosomes (e.g., copy number variants). Haplotype blocks and matched filters can be constructed for each segment of DNA to determine how many times each segment appears in a cell of interest, allowing, for example, the identification of CNVs when the number of segments is compared to that in a reference sequence. The systems and methods described herein could similarly be applied to mitochondrial DNA as well as nuclear DNA. As used herein, two nucleotide sequences may be considered “substantially identical” to each other if they can be effectively matched using the matched filtering methods disclosed herein. A nucleotide sequence may be substantially identical to another, for example, if the sequence is at least 95, 96, 97, 98, 99, 99.1, 99.2, 99.3, 99.4, 99.5, 99.6, 99.7, 99.8, 99.9, 99.91, 99.92, 99.93, 99.94, 99.95, 99.96, 99.97, 99.98, or 99.99 percent identical to the other. Shorter sequences may require higher similarity than longer sequences to be matched with the same confidence as the longer sequences.

With respect to a gamete, the haplotype blocks from which the unknown sequence are thought to be derived may be determined/measured by sequencing one or more cells that share the same genetic information as the gametocyte from which the gamete was derived (a cell comprising the same homologous chromosomes from which the gamete DNA is derived). For example, for a sperm cell of interest the candidate haplotypes may be measured/determined from one or more samples obtained from the male that made the sperm and/or for an ovum cell of interest the candidate haplotypes may be measured/determined from one or more samples obtained from the female that made the ovum. Similarly, the haplotype blocks from which an individual chromosome within a zygote, embryo, or individual organism, such as a human, may be derived may be determined by sequencing maternal and/or paternal cells from which the chromosome was inherited. Sequencing of a reference sample(s) obtained from an individual, or optionally related family members such as a mother and/or father, may be used to obtain the reference sequences for a gamete derived from the individual. Sequencing of a reference sample(s) obtained from an embryo's mother and/or father, or optionally other related individuals, may be used to determine the reference sequences for individual chromosomes within the embryo. The unknown sequences may then be reconstructed from the reference sequences according to the methods disclosed herein. The reference samples may be obtained by any suitable means known in the art. For example, cellular DNA may be obtained from blood (e.g., peripheral blood mononuclear cells (PBMCs)), saliva (e.g., buccal epithelial cells and white blood cells), hair follicles, and/or tissue biopsies. DNA may be obtained from somatic cells and/or suitable germline cells. According to some aspects of the disclosure, the known sequence(s) are determined (e.g., sequenced) before performing matched filtering to determine whether the known sequence(s) match the unknown sequence. Alternatively, the known sequence(s) may be determined after performing the matched filtering.

Measurements of the haplotype blocks may be made by sequencing, using suitable sequencing methodologies as is known in the art. Long-read sequencing may be preferable for capturing haplotype blocks. Long-read sequencing may be performed, for example, using single-molecule real-time sequencing (e.g., PACBIO® sequencing); nanopore sequencing (e.g., OXFORD NANOPORE® sequencing); and/or synthetic long-read or linked-read sequencing using technologies such as gel bead-in-emulsions (GEM) (e.g., 10× GENOMICS® sequencing), single-tube long fragment read (stLFR) sequencing (e.g., COMPLETE GENOMICS® sequencing), or transposase enzyme linked long-read sequencing (e.g., TELL-SEQ™ from UNIVERSAL SEQUENCING®.

A DNA sequence for an individual may also be phased using the genetic data of the parents of the individual and/or other related individuals, as is well known in the art, to determine candidate haplotypes for the individual's DNA (e.g., as may be passed on to the individual's gametes and offspring). Also, population data, such as data derived from the haplotype map database, HapMap, or the 1000 Genomes Project, may be used to phase individuals based on typical haplotypes found in the population. Software programs such as SHAPEIT and fastPHASE, or other methods that make use of Hidden Markov Models, are well known in the art and can be used for this approach. Accordingly candidate haplotype blocks may be determined by phasing a sequenced one or more reference samples.

Comparison of nucleotide-derived signals derived from DNA in the cell of interest and DNA in one or more reference samples may be used to determine whether various haplotypes measured in the reference samples are present in the DNA from a particular strand or chromosome within the cell of interest. As used herein, a “nucleotide-derived signal” may refer to any measurement that can be resolved for an individual strand or chromosome of DNA, that is at least somewhat indicative of the nucleotide composition of the DNA and that can be sufficiently correlated against a reference signal according to the methods described herein. According to some aspects of the disclosure, the signal may preferably be one that can be obtained through non-destructive means (without destroying and/or negatively impacting the health/viability of the cell of interest), such as various imaging modalities that are known in the art. Suitable imaging modalities may include, but are not limited to, digital microscopy/photography (e.g., using CCD or CMOS cameras), super-high resolution MM, and infrared spectroscopy (see, e.g., Mattson et al. Int. J. Mol. Sci. 2013, 14, 22753-22781, which is herein incorporated by reference in its entirety). Super high resolution MRI may preferably be performed with magnets greater than 10 Tesla. Imaging modalities such as X-ray may also be used, particularly where the health/viability of the cell of interest is of less importance. Fluorescent imaging modalities are also available for both live and dead cells in which fluorescent dyes may be used to stain DNA.

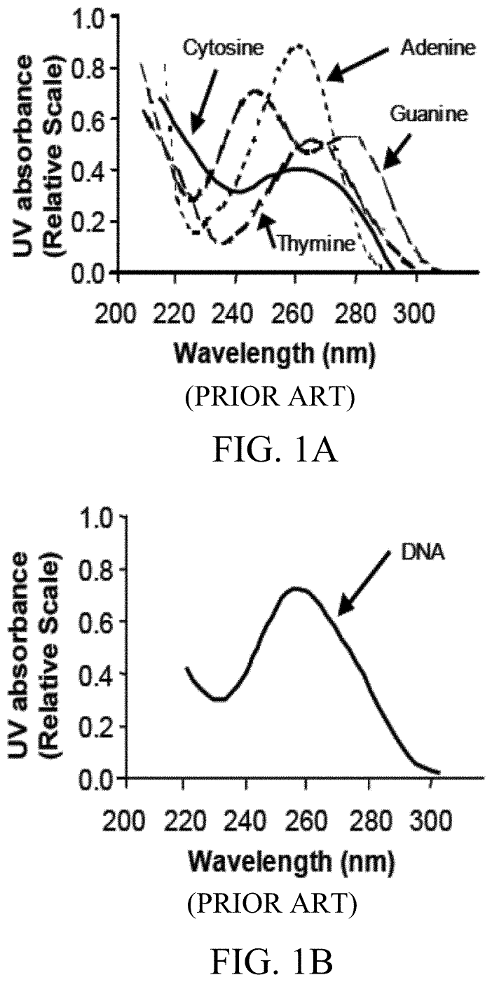

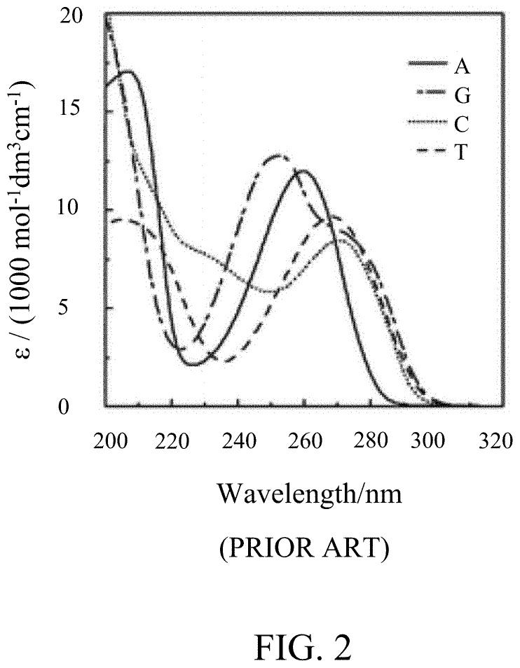

According to some aspects of the disclosure, the signal of interest obtained from the DNA in the cell of interest (the “input signal”) and the reference signals may be obtained via digital imaging, preferably high resolution imaging/photography, of the DNA in the cell of interest and reference sample(s). Digital imaging may be performed in combination with optical microscopy. The cell of interest and reference sample(s) may be illuminated with broad excitation light, such as white light, and/or narrow spectra of light, such as filtered light or laser. For example, the cell of interest and reference sample(s) may be illuminated and imaged by turning on and off a laser that is at frequencies where there is maximum difference in the absorption or emission coefficients of each of the nucleotides. FIGS. 1A-1B, reproduced from Otim, B. An investigation into the effectiveness of sunlight in disinfecting water from different sources [Thesis, NDEJJE University] 26 Jul. 2018; 10.13140/RG.2.2.20626.56009 (available on the world wide web at researchgate.net/publication/326624804_An_investigation_into_the_effectiveness_of_sunlight_i n_disinfecting_water_from_different_sources) and herein incorporated by reference in its entirety, illustrate that each of the four different nucleotides, adenine (A), cytosine (C), thymine (T), and guanine (G), have different absorption spectra, affecting the absorbance of a nucleic acid molecule. By way of example, images taken at the peak excitation of 262 nm may preferentially distinguish adenine since this nucleotide will exhibit substantial absorption of the photon energy at this wavelength whereas the other three nucleotides will exhibit approximately half the absorbance of adenine. As another example, images taken at peak excitation of 235 nm may minimize photon energy absorption by thymine, which would appear dark relative to the other three nucleotides. Similarly, images taken at peak excitation of 305 nm may maximize photon energy absorption by guanine relative to the other three nucleotides. Different conditions such as temperature and pH levels effect the absorption and emission spectra of the nucleotides and may be used to optimize images for the preferential detection of one or more nucleotides. For example, FIG. 2, reproduced from Kothekar, V. Module 10: Absorption spectroscopy of nucleic acids: DNA and RNA, Nucleic acid bases; Estimation of concentration, DNA purity, homogeneity. TECHNIQUES USED IN MOLECULAR BIOPHYSICS II (Based on Spectroscopy) (accessed on 28 Jan. 2022) (available on the world wide web at epgp.inflibnet.ac.in/epgpdata/uploads/epgp_content/S001174BS/P001858/M030435/ET/152663 9139P10_M10_ET.pdf) and herein incorporated by reference in its entirety, demonstrates that under different conditions, an image taken at peak excitation of approximately 290 nm would minimize absorption by adenine relative to the other nucleotides.

In embodiments in which the cell of interest is to remain healthy/viable, wavelengths may be selected in a manner which minimizes the probability for DNA damage caused by the incident light, such as single stranded and, more importantly, double stranded breaks. As described in Besaratinia, et al. FASEB J. 2011 September; 25(9): 3079-3091, herein incorporated by reference in its entirety, DNA damage is highly dependent on different wavelengths. Generally, wavelengths above about 300 nm do significantly less damage as they are absorbed less by DNA. For example, Sutherland et al., Radiat Res. 1981 June; 86(3):399-409, herein incorporated by reference in its entirety, indicates that the absorption spectrum of DNA is roughly 10E-3 to 10E-5 less in the range 330-370 nm than in the range of 250-300 nm. However, the less absorptive nature of this light can be compensated for by taking more images. In embodiments in which photons are typically absorbed in order for the photons to be re-radiated and the nucleotides to be visible, DNA fragmentation analysis, which is well understood in the art, at different wavelengths, different intensities of light, and different duration and numbers of images, may be used to optimize the imaging parameters for minimizing DNA damage while maximizing image resolution.

Furthermore, various techniques are known in the art preserving DNA integrity. For example, DNA-protective mediums, such as egg yolk, serum albumin, phospholipids extracted from lecithin, dimethyl sulfoxide (DMSO) and glycerol are well-known in the art. See, e.g., example Jeyendren et al. Fertil Steril. 2008 October; 90(4):1263-5 and Noda et al. Sci Rep. 2017 Aug. 17; 7(1):8557, each herein incorporated by reference in its entirety. Cells could be cryopreserved or simply bathed in a medium containing a DNA-protective medium such as dimethyl sulphide (DMS) or DMSO. Such techniques are commonly used in vitrification protocols and are found to have a protective effect on DNA damage at 2% and above. Many other substances that act as radical scavengers could also be used to protect the DNA from damage by Reactive Oxygen Species (ROS). Furthermore, cryopreservation may beneficially facilitate multiple images over an extended period of time, particularly in mobile cells, such as sperm. Also, fluorescent dyes, such as YOYO-1, for example, can be used to enable imaging of the DNA at lower wavelengths, less likely to cause damage.

According to certain aspects of the disclosure, multiple images may be taken of the cell of interest and/or reference sample(s). For instance, images may be taken under different imaging conditions such as at different wavelengths, different light intensities, different exposure times, different magnifications, and/or different angles as well as under different physiological conditions (e.g., temperature, pH) and over an extended period of time. By taking images in different colors, different nucleotides may be preferentially resolved. In some embodiments, multiple images may be constructively combined or summed into a single image/signal (e.g., images may be overlaid), through various methods known in the art. According to certain aspects of the disclosure, the DNA may preferably be imaged from three orthogonal axes so as to generate a three-dimensional image. If multiple images are taken over time of a mobile cell of interest (e.g., a sperm), each segment of interest in the DNA can be digitally aligned to efficiently convolve the signals. After aligning the segment in each image, the data on that segment could be constructively combined or summed over multiple images, to reduce noise before correlation. According to some aspects of the disclosure, images of the reference samples may be obtained under the same or similar imaging conditions. Images of the cell of interest and the reference samples may be taken using a singular imaging device. According to certain aspects of the disclosure, imaging of the DNA within the cell of interest and/or of the reference samples(s) may be performed in a manner that focuses on a particular segment of interest (e.g., a particular segment corresponding to a known haplotype). Such focusing may involve, for example, locating the segment of DNA, aligning the image to the segment of DNA, and/or magnifying a region of interest comprising the segment of DNA.

Matched filters can be used to compare each candidate haplotype block to an unknown sequence of interest. Various techniques of applying matched filters to signals are well known in the art, see, e.g., Matched Filter [Wikipedia entry] (available on the world wide web at en.wikipedia.org/wiki/Matched_filter (accessed on 28 Jan. 2022); Chen, Q. Matched filtering techniques, in Image Registration for Remote Sensing. 2011: 112-130 (Jacqueline Le Moigne, Nathan S. Netanyahu, & Roger D. Eastman eds.); Image registration based on the Fourier-Mellin transforms (available on the world wide web at thoduka.github.io/imreg_fmt/docs/fourier-mellin-transform/) (accessed on 28 Jan. 2022); Bancroft, J. C. Introduction to matched filters in CREWES Research Report. 2002; Volume 14; 8 pp. 8 (available on the world wide web at crewes.org/Documents/ResearchReports/2002/2002-46.pdf) (accessed 28 Jan. 2022), each of which is herein incorporated by reference in its entirety, and can be used to perform the matched filter constructed from a reference signal (e.g., image) on the signal (e.g., image) derived from the DNA in the cell of interest. In general, a “matched filter” refers any approach to comparing a known reference signal, or template, with an unknown/input signal to detect the presence of the template in the unknown/input signal. “Matched filtering” refers to applying one or more matched filters to the unknown/input signal to produce one or more output signals. In one embodiment, the comparison is done by convolving the unknown input signal with a conjugated reversed version of the template. Applying or comparing the matched filter to the input signal results in an output signal in which the amplitude is indicative of similarity with the reference signal.

Appendix I below illustrates this concept with example MATLAB code for convolving two possibly present haplotypes with a DNA signal. The DNA signal is simulated with four randomly chosen signal levels for each nucleotide, emulating for example the different levels of absorption of light at a particular wavelength by the 4 nucleotides, A, C, T and G. In addition, the DNA signal is convolved with a gaussian filter to emulate the blur that occurs when viewing nucleotides with a super high-resolution microscope. For example, with resolution of 50 nm, and approximate nucleotide length of 0.6 nm, the one-sided or “one-sigma” width of the gaussian blurring filter could be estimated at roughly 50/2/0.6=41.7 nucleotides. The code illustrates how a strand of imaged DNA can be analyzed by convolving it with two possible homologs to determine which homolog is present. A key aspect of this investigation is to see the difference in the convolution of two different homologs in practice, based on the conservative estimates of the genetic differences between the homologs. Many groups have described the factors that characterize genetic differences between people or between genetic homologs. For example, based on long-read sequencing of 42 unrelated individuals, it was estimated—see Shen at al. “High Coverage Whole-Genome Sequencing of Forty-Four Caucasians”, Plos One, Apr. 5, 2013, https://doi.org/10.1371/journal.pone.005949—that the average number a single nucleotide polymorphism (SNP) variants is roughly 3.3e6 per person, and the average number of indels if roughly 492,000 per person. Consequently, the separation between indels is on average 6Kb. An indel, which causes a shift between the reference homolog sequence and the imaged DNA sequence, is one of the important factors that causes sequence misalignment and reduces the correlation output. While indels vary from one to several hundred bases inserted or deleted, we make the generally conservative assumption in Appendix I that an indel is characterized by a deletion of a single base. This is conservative, because in the context of signal blurring as simulated by the Gaussian filter, one needs to have several bases deleted before one is outside the window of the Gaussian filter and consequently reduce the correlation between the imaged DNA and the reference homolog. We are further conservative in ignoring the effects of large copy number variants at the 1 kB level or more, which will cause the imaged DNA and the incorrect reference homolog to substantially decorrelate. The MATLAB code simulates SNPs and indels uniformly randomly distributed through the genome, with image blurring based on the imaging resolution, and the addition of normally distributed noise to the blurred image which has power or variance roughly ten times the variance of the signal produced by the imaged DNA due to the different fluorescence levels of the nucleotides. The noise power can be higher or lower relative to the signal power without changing the concept.

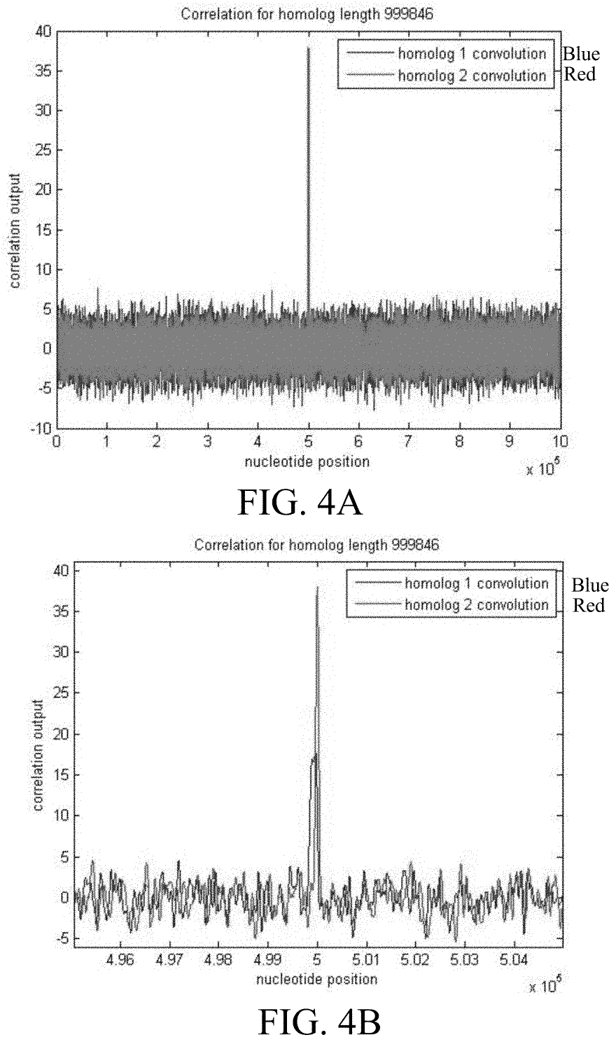

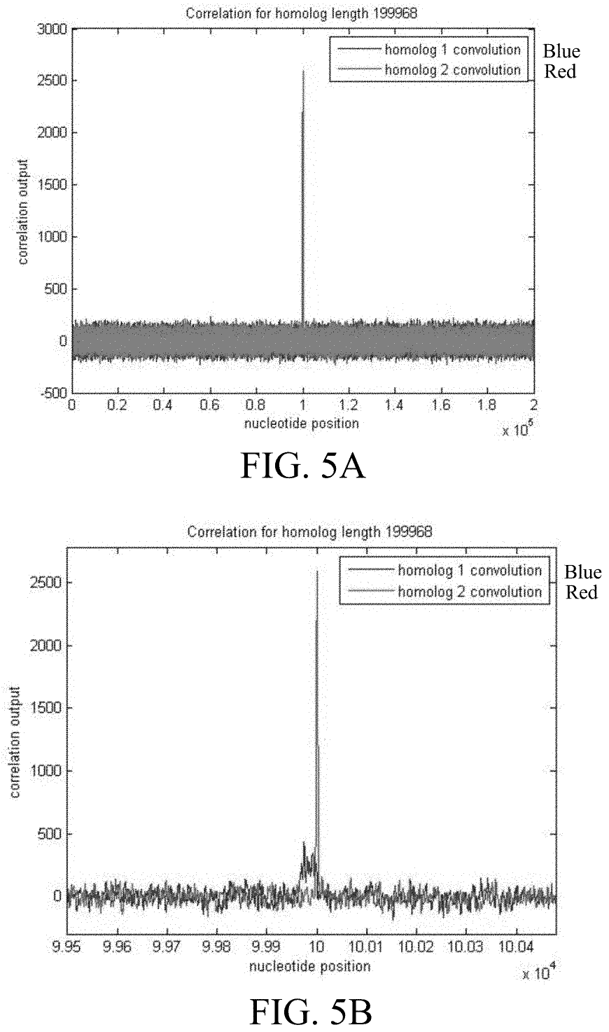

FIG. 4a illustrates the convolution output between the imaged DNA and two possible reference homologs in red (correct homolog) and blue (incorrect homolog), where resolution is approximately 50 nm (the Gaussian filter has one standard deviation of 41.6 nucleotides), the reference homolog length is 1 Mb, the target DNA is roughly twice the length of the reference hmologs, and the noise power is 10 times that of the signal. FIG. 4B zooms in on the peaks in FIG. 4A where the matching homolog fully aligns with the reference signal (red) the non-matching homolog (blue) partially aligns with the imaged DNA, until the series of single-nucleotide deletions reduces the correlation. FIG. 5A Shows the convolution output and correlation peaks for the matching (red) and non-matching (blue) homologs, where the resolution of the imaging is roughly 10 nm (with a standard-deviation on the Gaussian filter of roughly 8.3 nucleotides), the reference homologs are 200 kB in this context, the imaged DNA roughly double that size, and the noise power roughly ten times the signal power. FIG. 5B zooms in on the peaks in FIG. 5A where the matching homolog fully aligns with the reference signal (red) the non-matching homolog (blue) partially aligns with the imaged DNA, until the series of single-nucleotide deletions reduces the correlation.

Note that with the smaller resolution, one requires fewer deletions before the reference homolog and the imaged DNA decorrelate. One could also analyze the two or more reference homologs, to ensure that they have sufficient genetic differences to cause the wrong homolog to substantially decorrelate with the imaged DNA, based on the presence of indels, SNPS or structural variation such as CNVs, before applying them to the imaged DNA signal. Rather than being randomly chosen, as the MATLAB® code assumes, the reference homolog cutoff points can be chosen to ensure sufficient differences between the two reference homologs to cause substantially different correlations with the imaged DNA.

It can be shown based on the Cauchy-Schwarz inequality that the matched filter is the optimal linear filter for maximizing the signal-to-noise ratio (SNR) in the presence of additive stochastic noise. Matched filtering techniques are well known in the art for correlating two-dimensional and three-dimensional signals (e.g., images) such as images, as well as one-dimensional signals. In order to optimize the comparison of the input signal to the reference signal, the digital images may be rendered in forms that can be correlated, or convolved in the spatial domain, by following a trajectory of the strands of DNA. Image processing techniques for aligning two images are well known in the art and may include, for example, rotating the image mathematically. The mathematical approach to rotating an image in space involves two angles, such as phi and theta in spherical coordinates. It is well understood in the art how an information filtering technique, such as maximum-likelihood estimation or a Kalman filter, can be used to estimate the two angles describing the orientation of the DNA as one correlates the reference signal with the imaged DNA, stepping from one nucleotide to the next nucleotide, or one pixel in the digital image to the next. This is the same concept, for example, as simultaneously estimating the frequency and phase of an incident signal while correlating the incoming digitized signal against a reference code division multiple access (CDMA) signal in a CDMA receiver, such as in a Global positioning System (GPS) receiver. See for example Parkinson and Spilker, Global Positioning System: Theory and Applications, Volumes I & II, AIAA (Jan. 1, 1996), ISBN-,13: 978-1563471070.

Methods for evaluating matched filter outputs to assess correlation with the reference signal are well known in the art. The output signal of the matched filter can be quantified to provide an output value or quantitative measure of similarity between the DNA segment and the reference signal. For example, in some embodiments, the maximum value of the output signal (e.g., the peak of the convolution function) can be compared between the convolution functions generated by each of the reference homologs. The highest peak indicates the presence of the reference homolog in the imaged DNA. In some embodiments, one may track the correlation peak using a technique similar to the delay-locked loop used by CDMA receivers to track the correlation peak for an incident CDMA signal as one simultaneously estimates which is the correct matching CDMA code and estimates the phase of the signal by a phase-locked loop—see for example Parkinson and Spilker, Global Positioning System: Theory and Applications, Volumes I & II, AIAA (Jan. 1, 1996), ISBN-43: 978-1563471070. In the case of DNA imaging, rather than the phase of an incident signal, one is estimating one or two angles describing the alignment of the DNA on a plane or in three dimensions, while one matches the reference homolog “code” to the imaged DNA signal.

According to some aspects of the disclosure, the output value may be used to evaluate whether the reference signal is present in the input signal and, therefore, whether the haplotype from which the reference signal is generated is present in the DNA sequence being analyzed, with higher output values being indicative of a relatively increased probability that the reference signal is present and lower output values being indicative of a relative decreased probability that the reference signal is present. By applying the methods disclosed herein with reference signals to known DNA sequences that share and/or do not share the haplotype in a given reference signal, typical output values for each scenario can be determined and confidence intervals can be generated. Thresholds may be selected for determining whether output values are indicative of the presence or absence of the reference signal. The suitability of a particular threshold may be evaluated using Receiver Operating Characteristic (ROC) curve analysis, as is well understood in the art. See, e.g., Receiver operating characteristic [Wikipedia entry] (available on the world wide web at en.wikipedia.org/wiki/Receiver operating characteristic) (accessed on 28 Jan. 2022).

According to some aspects of the disclosure, it may be preferably to compare the input signal for each of one or more DNA segments in the cell of interest to multiple reference signals. For example, if all the possible haplotypes for a segment of DNA are known, then the input signal for that segment of DNA may be compared as described herein to reference signals for each of the possible candidate haplotypes. The haplotype present in the segment of DNA may be presumed to be whichever haplotype produces the higher output value (the strongest correlation). For instance, for a gamete, such as a sperm cell, haplotype blocks may be constructed from sequencing and phasing of diploid cells from the individual for each homologous chromosome, such that at each nucleotide within the DNA of unknown sequence the DNA may be presumed to belong to one of two haplotypes. Comparison of the input signal generated for a segment of the DNA comprising a given nucleotide may be used to determine which of the two haplotypes the nucleotide is derived from and, hence, the identity of the nucleotide. By using the methods described herein to evaluate the presence or absence of each haplotype block that forms a segment of DNA, the sequence of the DNA segment may be reconstructed based on the known sequences of the haplotype blocks.

The length of the matched filter may be determined, at least in part, by how high are the signal-to-noise ratio (SNR) and resolution of the image, how large are the haplotype blocks that are to be detected, and how well the input signal (e.g., the image of the chromosomal DNA in a cell of interest) can be aligned with the matched filter. According to some aspects of the disclosure, haplotype blocks may be constructed that are about or at least about 5, 10, 15, 20, 25, 30, 40, 50, 60, 70, 80, 90, 100, 150, 200, 250, 300, 350, 400, 450, or 500, 1000, 2000, 3000 kB in length depending on the resolution of the imaging technology. For example, the haplotype block(s) may be approximately 100 kB, in which case approximately 1000 matched filters could be constructed from a 100 Mb chromosome if one is covering each section of the imaged DNA by only one matched filter. However, one may create many matched filters to cover the same target DNA. By simply shifting by one nucleotide, we can create a new matched filter. According to some aspects of the disclosure, DNA within the cell of interest may be evaluated over segments of various and/or overlapping lengths (i.e. using matched filters of various or overlapping lengths). The digital signals obtained for the reference signal and input signal may be digitally truncated to a desired length/size to construct matched filters and compare signals to the matched filter. By evaluating the correlation between a reference signal and input signal at smaller length scales than the presumed haplotype blocks, errors in phasing and/or recombination events within a haplotype block may be identified. In some embodiments, the length of the matched filter may be approximately equal to the length of a haplotype block to be detected. In some embodiments, the length of the matched filter may be less than the length of the haplotype block to be detected (e.g., about 0.1, 0.2, 0.25, 0.3, 0.4, 0.5, 0.6, 0.7, 0.75, 0.8, 0.9 times the length of the haplotype block). In some embodiments, the length of the matched filter may be greater than the length of one or more haplotype blocks. For example, a matched filter may be constructed based on the combination of two or more haplotype blocks.

According to certain aspects of the disclosure, the signals (e.g., images) are obtained with the best possible resolution of the DNA in the nucleus or mitochondria. The size of a single nucleotide is approximately 0.6 nm, whereas the best resolution of photographic cellular images, depending on the situation and whether the cell can be dead or must be maintained alive and healthy, presently ranges from about 10 nm to about 1,000 nm, although the concepts disclosed herein apply at smaller levels of resolution as well. Some example techniques, termed super resolution microscopy (SRM) are described by Shermelleh at al, “Super-resolution microscopy demystified”, Nature Cell Biology, volume 21, pages 72-84 (2019). SRM can produce resolutions less than the traditional diffraction limit, which is limited roughly by the wavelength of the light. One method is stimulated emission depletion (STED) microscopy, which can tune resolution by adjusting laser power and can achieve resolutions in the range of 50-60 nm. This approach also has the capability using 3D STED to resolve images in the Z direction, providing the options to resolve between the lateral and axial resolution. Another method of breaking the diffraction limit is single-molecule localization microscopy (SMLM) which uses wide field illumination and switches by stochastic excitation to achieve single molecule switching and detection of fluorescent point emitters. This can be implemented on conventional, camera-based, wide-field imaging systems and can achieve resolution of around 20 nm lateral and 50 nm axial. While it is typically the case that high-resolution microcopy works better with fluorescent dyes added to the sample, the same concepts generally apply to samples that are illuminated and differentially absorb and re-emit photons, without added dyes. We expect that, with the diffraction limit broken, the field will continue to progress with increasingly high resolution and diminishing effect on the sample.

High resolution imaging/photography, as used herein may refer to imaging that can resolve sizes no greater than about 100,000, 90,000, 80,000, 70,000, 60,000, 50,000, 40,000, 30,000, 20,000, 10,000, 5,000, 1,000, 500, 100, 50, or 10 nm. Given that even the highest resolution imaging has not been able to detect and distinguish individual nucleotides, such imaging techniques have not been routine or conventional means for genotyping nucleic acids. However, as described herein, although the signal resolution (e.g., image resolution) may be orders of magnitude larger than the resolution of individual nucleotides such that the individual nucleotides may not be directly detected/imaged, what DNA haplotype is present in the input sequence can still be identified as a result of matched filtering, as described, and processing gain. As used herein, “processing gain” may be defined as the ratio of change in output to change in input. For example, assuming that that there are only two states of each digital data point because one of four nucleotides was illuminated and the others were not and that the input signal is compared to haplotype blocks of approximately 100 kB, the processing gain for aligning the matched filter for a haplotype block with the input signal would be the number of nucleotides in the DNA segment (100,000), or 10 log10(100,000)=50 kB. The processing gain would increase as each of the additional three nucleotides is able to be resolved, for example using different color lasers, rather than the presence/absence of only one nucleotide. By taking images in different colors, as described elsewhere herein, different nucleotides can be resolved.

### Implementation Systems

The methods described here can be implemented on a variety of systems. For instance, in some aspects the system for performing matched filtering includes one or more processors coupled to a memory, such a computing device. The methods can be implemented using code and data stored and executed on one or more electronic devices. Such electronic devices can store and communicate (internally and/or with other electronic devices over a network) code and data using computer-readable media, such as non-transitory computer-readable storage media (e.g., magnetic disks; optical disks; random access memory; read only memory; flash memory devices; phase-change memory) and transitory computer-readable transmission media (e.g., electrical, optical, acoustical or other form of propagated signals—such as carrier waves, infrared signals, digital signals).

The memory can be loaded with computer instructions to perform the matched filtering. The memory can be loaded with one or more signals for performing the matched filtering (e.g., input signal(s), reference signal(s). Various signals (e.g., images) may be stored on one or more databases within the memory. In some aspects, the system is implemented on a computer, such as a personal computer, a portable computer, a workstation, a computer terminal, a network computer, a supercomputer, a massively parallel computing platform, a television, a mainframe, a server farm, a widely-distributed set of loosely networked computers, or any other data processing system or user device.

The methods may be performed by processing logic that comprises hardware (e.g. circuitry, dedicated logic, etc.), firmware, software (e.g., embodied on a non-transitory computer readable medium), or a combination of both. Operations described may be performed in any sequential order or in parallel.

Generally, a processor can receive instructions and data from a read only memory or a random access memory or both. A computer generally contains a processor that can perform actions in accordance with instructions and one or more memory devices for storing instructions and data. Generally, a computer will also include, or be operatively coupled to receive data from or transfer data to, or both, one or more mass storage devices for storing data, e.g., magnetic disks, magneto optical disks, optical disks, or solid state drives. However, a computer need not have such devices. Moreover, a computer can be embedded in another device, e.g., a smart phone, a mobile audio or media player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including, by way of example, semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

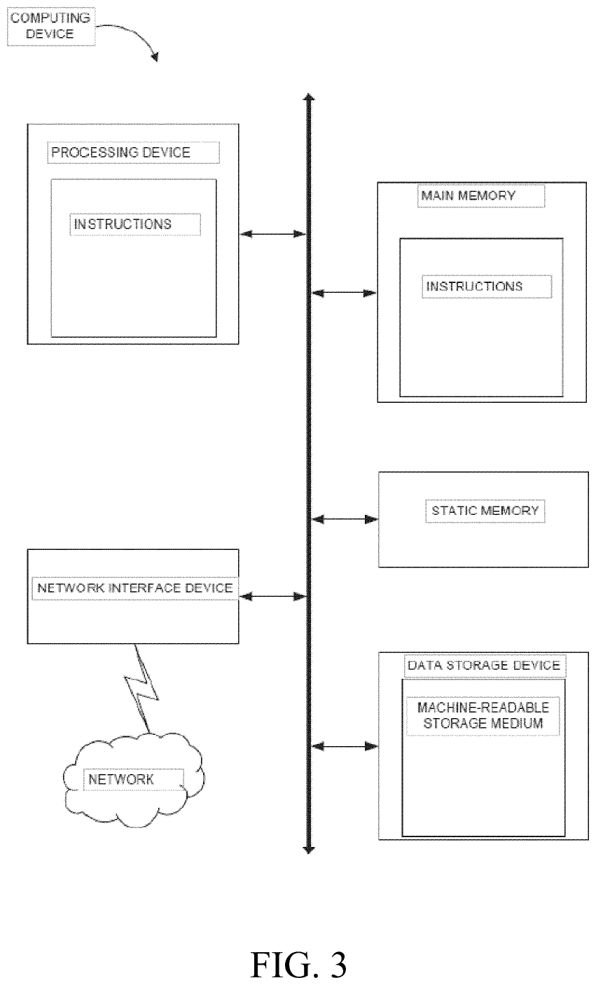

An exemplary implementation system is set forth in FIG. 3. Such a system can be used to perform one or more of the operations described here. The computing device may be connected to other computing devices in a LAN, an intranet, an extranet, and/or the Internet. The computing device may operate in the capacity of a server machine in client-server network environment or in the capacity of a client in a peer-to-peer network environment. In some embodiments, one or more imaging apparatuses or devices or components thereof (e.g., a camera) for obtaining signals for use in the methods described herein may be operably connected (e.g., over a network, via a direct wired connection) to a computing device for performing the matched filtering. The system may facilitate or automate the transfer of signals such as images between the memory of the computing device and the imaging apparatuses or devices. The implementation system may further include various means known in the art (e.g., monitors) for outputting (e.g., displaying) input signals, reference signals, output signals, output values, and/or results of a correlation described herein (e.g., a nucleotide sequence for a segment of DNA).

### Applications

Various potential applications may arise from correlating signals for DNA within a cell to signals for reference samples. The genotyping arising from the correlations may be used in any suitable application as is known in the art. The determination of ploidy status or identification of CNVs in a portion of DNA of a cell of interest, as described elsewhere herein, may be used for example, in any of the methods that similarly rely on a determination of ploidy status or identification of CNVs that are disclosed in international PCT application PCT/US2021/057400 to Kumar et al. filed on Oct. 29, 2021, which is herein incorporated by reference in its entirety. Described herein are several specific, but non-limiting, examples of how such correlations can be used to drive subsequent decisions and/or further analysis or treatments.

### Screening Genotypes

The methods described herein may be used to genotype the cell of interest, partially or entirely (e.g., detect genetic variants in a cell of interest). The methods described herein may be used to detect inherited variants (i.e. a variant at one or more loci of one of a subject's chromosomes inherited from a parent) or de novo variations, such as chromosomal structural variants (e.g., CNVs) relative to the structure in the corresponding chromosomal homologue or haplotype of the parent or parent cell from which the chromosomal homologue or haplotype was inherited. The variant(s) of interest may be associated with a disease, condition, or other disorder. The variant may, for example, be causative of a disease or otherwise associated with a prevalence or increased likelihood of disease (e.g., an increased susceptibility to a disease). Examples of specific associations between variants and disease are well known in the art. The variant(s) of interest may be associated with other traits or phenotypes (e.g., eye color), particularly as may be desirable to screen for in potential offspring. The analysis of the genome for a subject of interest for which the cell of interest is evaluated or for genetic relatives (e.g., the father and/or mother) of a subject (e.g., where the subject is an embryo) may be used to identify potential variants for subsequent analysis in the subject.

According to some aspects of the invention, a determination of the parent of origin of the haplotype having a variant may be made. Such determinations may be possible, for example, based on the phasing of the variant. Additional sequencing may be performed on one (the originating parent) or both of the parents to confirm the determination. For example, whole genome sequencing (e.g., shotgun sequencing) may be performed on the parent(s), which may allow confirmation of the corresponding variant in the originating parent.

According to some aspects of the invention, the genotyping (e.g., the determination of the presence or absence of one or more variants) may be used to inform decisions related to assisted reproductive technology (ART), including procedures involving artificial insemination (e.g., intracervical insemination (ICI) or intrauterine insemination (IUT)) or in vitro fertilization (IVF), which are well known in the art. According to certain aspects of the disclosure, the cell of interest is a gamete. In one embodiment, potential gametes (e.g., sperm or ova) for ART (e.g., IVF) are screened based on genotype. Because the methods disclosed herein comprise non-destructive techniques for genotyping, they may be particularly suitable for screening candidate gametes for use in ART. The methods described herein may be used to select one or more gametes for use in ART (e.g., IVF) and/or to select one or more gametes (e.g., ova) for discarding/disposal. The methods may be used to select one or more gametes for freezing (e.g., for future use in ART). For example, a determination of risk of disease may be made for an embryo at least in part based on the detection of variant or chromosomal abnormality in a gamete to be used for ART. In some implementations, a gamete with no identified disease-associated variants may be selected for implantation or freezing. In some implementations, any gamete with an identified disease-associated variant may be disposed of or discarded. In some implementations, the available gametes (e.g., ova) may be ranked based entirely or at least in part on the identification of variants (e.g., by the number of disease-associated variants and/or the presence of particular variants). The same determinations may be made with respect to variants associated with other traits or phenotypes that are not disease-associated. The variant may be singularly associated with a trait, disease, or other phenotype (a Mendelian trait) or may be associated with a higher frequency of a trait, disease, or other phenotype, as through a polygenic model. The determinations made from the methods described herein may be used independently or in combination with existing methods of screening for ART, as is well known in the art.

For example, if the DNA of the father that makes a sperm is able to be sequenced, once the haplotype blocks that are present in the sperm are known by a matched filtering technique, the exact DNA that is in the sperm may be determined. The genotypes of candidate sperm cells can then be used, for example, to select sperm to fertilize eggs in order to minimize disease probability or maximize certain traits in the children, based on single gene phenotype associations or polygenic predictive modeling, as is well known in the art. See, e.g., WO2020/191195 to Rabinowitz; WO2020/198732 to Rabinowitz; WO2021/067417 to Kumar et al., each of which is herein incorporated by reference in its entirety. For instance, hundreds of sperm could be imaged to select one sperm cell for fertilization by Intra-Cytoplasmic Sperm Injection (ICSI). All sperm candidates from the same male could be evaluated using the methods described herein with the same matched filters.

According to specific aspects of the invention, the cell of interest may be from an embryo or a fetus. As used herein, an “embryo” may refer to a cellular organism produced by sexual reproduction, including a zygote, morula, and blastocyte, up to the stage of development where the embryo becomes a fetus. An embryo, may exist in vitro (e.g., for purposes of IVF) or in utero. As used herein, a “fetus” may refer to an unborn offspring produced by sexual reproduction and existing in utero, beginning at the stage of development where the unborn offspring is no longer characterized as an embryo. Thus, a subject may be considered either an embryo or a fetus from the single cellular stage until the fetus is born. In humans, the offspring is usually considered to be a fetus at approximately 8 weeks following conception. The cell of interest may be isolated from the embryo or fetus or imaged in situ within the embryo or fetus. The techniques and inherent risks for obtaining cellular samples from an embryo or fetus are well understood in the art. According to some aspects, cellular DNA may be obtained from a biopsy of an embryo or fetus, as is known in the art.

The methods described herein may be performed on a single embryo or on a plurality of embryos (e.g., a plurality of embryo candidates for implantation). The methods described herein may be used to select one or more embryos for implantation and/or to select one or more embryo's for discarding/disposal. The methods may be used to select one or more embryo's for freezing (either in the case that the embryo is selected for possible future implantation or in the case that the embryo is not a primary candidate for implantation but it is not desired to be disposed of). For example, a determination of risk of disease may be made for an embryo at least in part based on the detection of variant or chromosomal abnormality in the cell of interest (e.g., the identification of a CNV, particularly one having a known association with a disease). In some implementations, an embryo with no identified disease-associated variants may be selected for implantation or freezing. In some implementations, the embryos may be ranked based entirely or at least in part on the identification of variants (e.g., by the number of disease-associated variants and/or the presence of particular variants). The determinations made from the methods described herein may be used independently or in combination with existing methods of preimplantation genetic testing (PGT), as is well known in the art.

According to some aspects of the invention, the genotyping or other determinations made via the methods described herein may be used to inform decisions on pregnancy, particularly where the subject is a fetus. For example, the decision whether to continue or terminate a pregnancy may be based on the genotyping (e.g., the identification of a variant or chromosomal abnormality) in the same manner as decisions are made regarding IVF, as described elsewhere herein. The determinations made from the methods described herein may be used independently or in combination with existing methods of prenatal diagnosis, as is well known in the art.

According to certain aspects of the invention, the genotyping may be used to inform additional testing and/or methods of diagnosis. For example, upon the identification of a disease-associated variant, additional PGD or prenatal diagnostic testing may be ordered. In some instances, the additional testing may be specific to one or more diseases associated with the variant detected. In some instances, more invasive procedures may be performed on the subject, particularly if the subject is an embryo or fetus. For example, tissue biopsies may be performed directly on the embryo or fetus in order to perform sequencing of NA or other diagnostics on the cellular material. Karyotyping may be performed on the subject. In some implementations, the additional testing may be performed substantially concurrently with performance of the methods described herein (at approximately the same level of development). In some implementations, additional testing may be performed on a postponed schedule, allowing for additional development to occur (e.g., for development from an embryo to a fetus and/or after implantation of an embryo via IVF). In some implementations, additional testing may be performed on a born subject (e.g., an infant or child subject) based on genotyping performed when the subject was an embryo and/or fetus.

According to certain aspects of the invention, the genotyping may be used to inform treatment decisions for the subject. For example, upon the identification of a variant, the subject may be treated for a disease or condition associated with the variant. The treatment may comprise any treatment suitable for the subject's stage of development. For example, gene editing may be performed on an embryo and/or prenatal treatments may be administered to a fetus (or mother carrying the fetus). In some implementations, treatments may be performed on a postponed schedule, allowing for additional development to occur (e.g., for development from an embryo to a fetus and/or after implantation of an embryo via IVF). In some implementations, treatment may be performed on a born subject (e.g., an infant or child subject) based on genotyping from when the subject was an embryo and/or fetus. The early detection of a variant (e.g., while in utero) may allow for earlier treatment in infants and children, which may provide improved outcomes.

Genetically Profiling Tumors having Chromosomal Instability

Genomic instability of tumor cells is often associated with poor patient outcome and resistance to targeted cancer therapies. The accumulation of genetic and epigenetic lesions in response to environmental exposures to carcinogens and/or random cellular events often results in the inactivation of tumor suppressor genes that play critical roles in the maintenance of cell cycle, DNA replication and DNA repair. Loss or inhibition of cellular DNA repair mechanisms often results in an increased mutation burden and genomic instability. CNVs are prevalent across many types of cancer types and may cause the gain of oncogenes and/or loss of tumor suppressors associated with disease progression and therapeutic response or resistance. Genomic instability is associated with sub-clonal heterogeneity and is frequently observed in solid tumors between different lesions, within the same tumor, and even within the same solid biopsy site. Such tumor cell heterogeneity can complicate therapeutic intervention designed around single molecular targets. Genome-wide CNV profiles can be used to characterize genomic instability, However, assessment of genomic instability in bulk tumor or biopsy can be complicated due to sample availability as well as noise stemming from surrounding tissue contamination or tumor heterogeneity. Tumors associated with increased genomic instability have been shown to respond to specific types of therapies, including, for example, platinum-based chemotherapy and PARP inhibitors. See, e.g., Greene et al., PLoS One. 2016 Nov. 16; 11(11):e0165089 (doi: 10.1371/journal.pone.0165089), which is herein incorporated by reference in its entirety.

Poly ADP ribose polymerases (PARPs), nuclear enzymes found in almost all eukaryotic cells, catalyze the transfer of ADP-ribose units from nicotinamide adenine dinucleotide (NAD+) to nuclear acceptor proteins, and are responsible for the formation of protein-bound linear and branched homo-ADP-ribose polymers. Activation of PARP and resultant formation of poly(ADP-ribose) can be induced by DNA strand breaks after exposure to chemotherapy, ionizing radiation, oxygen free radicals, or nitric oxide (NO). Several forms of cancer are more dependent on PARP than regular cells, making PARP an attractive target for cancer therapy, independent of the specific cancer indication. Also, because PARP is associated with the repair of DNA strand breakage in response to DNA damage caused by radiotherapy or chemotherapy, it can contribute to the resistance that often develops to various types of cancer therapies. Consequently, inhibition of PARP may retard intracellular DNA repair and enhance the antitumor effects of cancer therapy. Indeed, in vitro and in vivo data show that many PARP inhibitors potentiate the effects of ionizing radiation or cytotoxic drugs such as DNA methylating agents. The PARP family of enzymes is extensive and competitive inhibitors of PARP are known. Approved PARP inhibitors include olaparib (Lynparza®, AstraZeneca); rucaparib (Rubraca®, Clovis Oncology); niraparib (Zejula®, Tesaro); and talazoparib (Talzenna®, Pfizer). Other PARP inhibitors being studied include veliparib (ABT-888, AbbVie); pamiparib (BGB-290) (BeiGene, Inc.); CEP 9722 (Cephalon); E7016 (Eisai); and 3-aminobenzamide. Platinum-based chemotherapeutic (antineoplastic drugs, informally called “platins”) are coordination complexes of platinum, including cisplatin, oxaliplatin, and carboplatin, as well as several proposed drugs under development. Platinum-based chemotherapeutics cause crosslinking of DNA as monoadduct, interstrand crosslinks, intrastrand crosslinks or DNA protein crosslinks that inhibits DNA repair and/or DNA synthesis.

Other forms of treatment that are appropriate for cancers exhibiting chromosomal instability are understood in the art. As described elsewhere, the methods disclosed herein may be used to identify CNVs in a cell of interest, such as a cancer cell or tumor cell (e.g., a circulating tumor cell (CTCs)). Accordingly, the methods described herein may relate to identifying genetic signatures in subjects having cancer that are indicative of chromosomal instability and, therefore, suitable for classes of therapeutics targeting genetic mechanisms (e.g., inhibiting the repair of DNA so that the damaged DNA may be more effectively targeted). These therapeutics may be agnostic to the specific type of cancer. Accordingly, the methods described herein may be performed on subjects diagnosed as having or suspected of having cancer prior to or concurrently with specific cancer diagnoses and/or tissue biopsies. The genetic analysis described herein may be performed concurrently with other routine analyses and/or cancer diagnoses or assessment based on the same or different biological samples collected at the same time.

While the invention has been described and exemplified in sufficient detail for those skilled in this art to make and use it, various alternatives, modifications, and improvements should be apparent without departing from the spirit and scope of the invention. The examples provided herein are representative of preferred aspects, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention. Various aspects of the invention will be understood to be combinable unless not physically possible or otherwise indicated by context.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred aspects and optional features, modification and variation of the concepts herein disclosed may be resorted to by those skilled in the art, and that such modifications and variations are considered to be within the scope of this invention as defined by the appended claims.

APPENDIX I
% matched_filter_correlation.m
% simulates imaged DNA and reference homologs to generats the
% correlation function for a matched filter to identify
% which homolog is present in the DNA
% constants

| | |
|---|---|
| num_b = 3e9; | % number nucleotides in the genome |
| num_SNP = 3.3e6; | % average number of SNP variants |
| num_indel = 492e3; | % average number of indel variants |
| sig_lev = rand(1,4); | % randomly select signal levels for each |

nucleotide assuming single color laser

| | |
|---|---|
| dna_len = 200000; | % approximate length of reference homolog |

| | |
|---|---|
| dna_len_fact = 1;10 | % additional amount of DNA of length |

dna_len added to the imaged dna, beyond one of the homologs

| | |
|---|---|
| sigma = 2/0.6/2; | % 1-sigma measured in terms of nucleotides |

(0.6nm) of the gaussian filter

| | |
|---|---|
| len_g = 200; | % length of the Gaussian filter in terms |

of nucleotides

| | |
|---|---|
| noise_pow_fact = 10; | % added gaussian noise will have this |

much more variance than the signal variance
% computed constants
len_SNP = round(num_b/num_SNP); % mean separation between SNPS
in bases
len_indel = round(num_b/num_indel); % mean separation between
indels in bases
% forming first homolog
hom1 = randint(1, dna_len, [1 4]); % integers 1-4 represent
different nucleotides A,C,T,G assumed uniformly distributed
% forming second homolog
hom2 = hom1; % start with hom2 matching hom1 then edit in the
random differences
% adjusting for SNPS
ind_SNP_tmp = round(rand(floor(dna_len/len_SNP),1)*len_SNP*2); %
find the random spacing between SNP variants
ind_SNP_tmp = filter(1, [1 −1], ind_SNP_tmp); % integrate to
find indexes of the SNP variants in hom2
ind_SNP = ind_SNP_tmp(find(ind_SNP_tmp <= dna_len)); % drop end
if we happen to have indexes out of range dna_len
hom2(ind_SNP) = randint(1, length(ind_SNP), [1 4]); % insert
different nucleotides at those SNP indexes
% adjusting for indels
ind_indel_tmp =
round(rand(floor(dna_len/len_indel),1)*len_indel*2); % find the
random spacing between SNP variants
ind_indel_tmp = filter(1, [1 −1], ind_indel_tmp); % integrate to
find indexes of the indels variants in hom2
ind_indel = ind_indel_tmp(find(ind_indel_tmp <= dna_len)); %
drop end if we happen to have indexes out of range dna_len
ind = [1:dna_len]; % start with index for all positions in hom1
ind_del = setdiff(ind, ind_indel); % find those indexes which
are preserved after single nucleotid deletions
% drop single nucleotides -- this is generally conservative
since usually
% indels involve 1 to hundreds of nucleotides, and cause more
difference in
% the correlation between hom1 and hom2. Rare cases, however,
could be X
% base deletion followed by X base insertion, that would cuase
hom1 and
% hom2 to match more closely than two single base deletions in
succession
hom2 = hom2(ind_del);
% scaling
hom_len = min(length(hom1), length(hom2)); % make same length
hom1r = hom1(1:hom_len);
hom2r = hom2(1:hom_len);
% creating reference signals levels matching the imaged DNA
signals
% (without noise and blurring)
hom1rs = sig_lev(hom1r);
hom1rs = hom1rs-mean(hom1rs);
hom2rs = sig_lev(hom2r);
hom2rs = hom2rs-mean(hom2rs);
% simulate imaged signal including only homolog 2 i.e. only hom2
is acutally present in the DNA
% (we could just as easily inlcude only homolog 1)
ima = [randint(1, dna_len/2*dna_len_fact, [1 4]) hom2 randint(1,
dna_len/2*dna_len_fact, [1 4])]; % add random nucleotides assumed
unformly distributed

| | |
|---|---|
| imas = sig_lev(ima); | % convert to fluorescence levels |
| imas = imas-mean(imas); | % substract the mean of the imaged |

```
signal
ima_len = length(imas);
% blurring signal with a gaussian filter
x_tmp = [−len_g:2*len_g/(len_g−1):len_g];
gauss = exp(−x_tmp.\hat{ }2/(2*sigma\hat{ }2));
gauss = gauss/(sum(gauss));
plot(x_tmp, gauss);
% blurring signal to emulate level of resolution based on
Gaussian filter
imash = cconv(imas, gauss, ima_len);
hom1rsh = cconv(hom1rs, gauss, hom_len); % can also blurr
reference to match disortion of image DNA
hom2rsh = cconv(hom2rs, gauss, hom_len);
% adding noise
imashn = imash +
randn(1,ima_len)*std(sig_lev)*sqrt(noise_pow_fact); % additive
normally distributed noise after image blurring
% convolving using circular convolution with reversed signal.
Many other
% approaches possible that leverage same matched filtering
concept
cconv1 = cconv(hom1rsh(end:−1:1), imashn, hom_len);
cconv2 = cconv(hom2rsh(end:−1:1), imashn, hom_len);
figure;
plot(cconv1);
hold on;
plot(cconv2,′r′);
xlabel(′nucleotide position′);
ylabel(′correlation output′);
title([′Correlation for homolog length ′ num2str(hom_len)]);
legend(′homolog 1 convolution′, ′homolog 2 convolution′)
cconv1_max = max(cconv1);
cconv2_max = max(cconv2);
if cconv2_max > cconv1_max
  fprintf(′Able to resolve correct homolog 2 present in imaged
DNA′)
else
  fprintf(′Too much noise or blurr. Make homologs longer for
more processing gain′)
end
```

1. A method of genotyping a segment of DNA, the method comprising:
obtaining a signal derived from the DNA segment that is indicative of the nucleotide composition of the DNA segment;
comparing the signal to one or more reference signals derived from different reference samples of DNA with matched filtering; and
determining based on the matched filtering whether the nucleotide sequence of the DNA segment is substantially identical to a nucleotide sequence within one of the one or more reference samples.

2. The method of claim 1, wherein determining whether the nucleotide sequence of the DNA segment is substantially identical to a nucleotide sequence of one of the one or more reference samples comprises determining whether the DNA segment comprises the same haplotype block as the nucleotide sequence of one of the one or more reference samples.

3-5. (canceled)

6. The method claim 2, wherein the haplotype block(s) are at least about 100 kB in length.

7. The method of claim 1 any one of the preceding claims,
wherein comparing the signal to one or more reference signals comprises comparing the signal to two reference signals, each of the two reference signals being derived from two different but homologous chromosomes, and
wherein determining based on the matched filtering whether the nucleotide sequence of the DNA segment is substantially identical to a nucleotide sequence of one of the one or more reference samples comprises determining which of the two homologous chromosomes the DNA segment is derived from by determining which reference signal produces the highest output value from the matched filtering.

8. (canceled)

9. The method of claim 1,
wherein comparing the signal to one or more reference signals comprises comparing the signal to four reference signals, each of the four reference signals being derived from four different but homologous chromosomes,
wherein determining based on the matched filtering whether the nucleotide sequence of the DNA segment is substantially identical to a nucleotide sequence of one of the one or more reference samples comprises determining which of the four homologous chromosomes the DNA segment is derived from by determining which reference signal produces the highest output value from the matched filtering,
wherein the DNA segment comprises a segment of a chromosome from a diploid cell of an organism and two of the four reference signals are derived from a mother of the organism and two of the four reference signals are derived from a father of the organism, and
wherein the diploid cell is an embryonic cell.

10-11. (canceled)

12. The method of claim 1, wherein comparing the signal to one or more reference signals with matched filtering comprises convolving the signal with conjugated reversed versions of each of the one or more reference signals.

13-20. (canceled)

21. The method of claim 1, wherein the signal was derived from the DNA segment in a live cell.

22. The method of claim 2, wherein the haplotype blocks of the one or more reference samples were determined using long-read sequencing, synthetic long-read sequencing, or phasing based on parent genomes or population data.

23. The method of claim 1, further comprising assigning a nucleotide sequence to the DNA segment based on the nucleotide sequences of the one or more reference samples or determining a copy number of the DNA segment or of a portion of DNA with the DNA segment.

24-26. (canceled)

27. A method of screening a plurality of gamete cells, the method comprising:
performing the method of claim 1 on DNA within the plurality of gamete cells, and
selecting and isolating a gamete cell based on a genotype of one or more segments of DNA within the gamete cell.

28. The method of claim 27, wherein the selected gamete cell is disposed of or discarded, frozen, or used in assisted reproduction.

29-31. (canceled)

32. The method of claim 28, wherein selecting a gamete cell based on a genotype of one or more segments of DNA within the gamete cell comprises generating one or more phenotype predictive models from the genotype.

33. A method of screening a plurality of embryos, the method comprising:
performing the method of claim 1 on DNA within at least one cell from each of the plurality of embryos, and
selecting and isolating an embryo based on a genotype of one or more segments of DNA within the embryo.

34. The method of claim 33, wherein the selected embryo is disposed of or discarded, frozen, or used in assisted reproduction.

35-36. (canceled)

37. A method of detecting chromosomal instability in tumor DNA, the method comprising:
performing the method of claim 1 on DNA within a tumor or cancer cell to determine a ploidy status for one or more chromosomal segments within the tumor or cancer cell, wherein identification of an aneuploidy status for the one or more chromosomal segments is used to indicate chromosomal instability of at least some tumor cells, and
treating the cancer or tumor cell or a subject from which the cell was obtained for cancer based on whether chromosomal instability has been indicated.

38-39. (canceled)

40. The method of claim 37, wherein the treatment comprises administering poly ADP ribose polymerase (PARP) inhibitors or platinum-based chemotherapeutics if chromosomal instability is indicated.

41. (canceled)

42. A method of generating signals indicative of the nucleotide composition of a segment of DNA within a cell of interest, the method comprising:
imaging the segment of DNA within the cell of interest; and
imaging one or more homologous segments of DNA within one or more reference cells.

43. (canceled)

44. The method of claim 42, wherein images obtained from imaging the segment of DNA and one or more homologous segments of DNA are three-dimensional images.

45. The method of claim 42, wherein imaging the segment of DNA and one or more homologous segments comprises illuminating the DNA with one or more wavelengths of light that each preferentially distinguishes a different type of nucleotide.

46-47. (canceled)

48. The method of claim 42, wherein the imaging of the segment of DNA and the one or more homologous segments of DNA is performed with a single imaging apparatus.

49-54. (canceled)
